(19) United States
(12) Patent Application Publication
(10) Pub. No.: US 2009/0312413 A1
(43) Pub. Date: Dec. 17, 2009

(54) Composition Comprising Tanshinone Compounds Isolated From The Extract Of Salviae Miltiorrhizae Radix For Treating Or Preventing Cognitive Dysfunction And The Use Thereof

(75) Inventors: Sang Seob Song, Gyeonggi-do (KR); Young Ho Kim, Gyeonggi-do (KR); Jong Moon Kim, Seoul (KR); Hee Kim, Gyeonggi-do (KR); Hee Jin Ha, Gyeonggi-do (KR); Hoon Yi Jung, Seoul (KR)

Correspondence Address: KIRK HAHN, 14431 HOLT AVE, SANTA ANA, CA, 92705, US

(73) Assignees: DIGITAL BIOTECH CO., LTD., Gyeonggi-do (KR); ILSUNG PHARMACEUTICALS CO., LTD., Seoul (KR)

(21) Appl. No.: 12/089,048

(22) PCT Filed: Oct. 4, 2006
(86) PCT No.: PCT/KR2006/003999

§ 371 Date: Apr. 2, 2008

(30) Foreign Application Priority Data
Oct. 6, 2005 (KR) 10-2005-0093877

(51) Int. Cl. A61K 31/343; A61K 31/122; A61P 25/00; A61P 25/28; A61P 25/16

(52) U.S. Cl. 514/468; 514/680

(57) ABSTRACT

A composition comprising tanshinone compounds selected from the group consisting of miltirone, 1,2-didehydromiltirone, tanshinone IIA, tanshinone I and dihydrotanshinone I isolated from Salviae miltiorrhizae Bunge showing potent inhibiting effect on the aggregation and toxicity of beta-amyloid and recovering activity of memory learning disorder confirmed by Y maze study and Passive Avoidance study. Therefore it can be used as the therapeutics or health food for treating and preventing cognitive function disorder with safe.

### TECHNICAL FIELD

The present invention relates to a composition comprising the fraction of Salviae miltiorrhizae showing preventive and treating activity of cognitive dysfunction disease and the use thereof.

### BACKGROUND ART

CNS (Central Nervous System) consisting of brain and spinal cord which plays a main role in regulating life phenomenon is a essential organ governing all the human function through from sensory and (in)voluntary movement to thinking, memory, motion, language etc. Accordingly, a rapidly progressed apoptosis of neuronal cell caused by stroke, trauma etc as well as slowly progressed apoptosis such as degenerative disease occurring in CNS caused by senile dementia for example, Alzheimer's disease or Parkinson disease etc result in irreversible functional disorder of neuronal network, which give rise to immortal failure of human function in the end. Among them, the patients suffering from Alzheimer disease, a representative senile dementia have been increased in proportion to both of extended life-span and modernized welfare facility. According to the public survey of Korea Institute for Health and Social Affair, the ratio of older people among Korean people exceeds 7% in 2000, reaches to 8.3% (3,970,000) and shall approach to 14.4% in 2019. Especially, the ratio of more than 65 years old patient suffering with senile dementia is presumed to 8.2% in Korea. In Western countries, about 10% among more than 65 years old and about 40-50% among 80 years old patient suffers with senile dementia. Since more than five million patients suffer with the disease, the medical expense caused thereby is presumed to hundred billion dollars in a year. There have been found that more than about two hundred thousand people are suffering from dementia in Korea. In America, it has been presumed the number of the patients be increased to two fold than the number of present patients in 2030 and fourteen million (more than 350%) in 2050.

Since Alzheimer's disease initiated with cognitive function disorder is one of long-term degenerative diseases resulting in the breakdown of human nature, there have been tried to develop effective and preventive drugs till now, for example, acetylcholinesterase inhibitor such as Aricept® (Pfizer Co.), Exelon® (Novartis Co.), Reiminyl® (Janssen Co.) or NMDA receptor antagonist such as Ebixa® (Lundbeck Co.). However, the acetylcholine esterase inhibitor could just alleviate reduced cognitive ability and could not satisfactorily treat etiological cause of the disease. Although the drug shows temporarily alleviated effect on only some of patients (about 40-50%), it could not maintain it's potency for a long time moreover it shows various adverse response such as hepato-toxicity, vomiting, anorexia in case of long-term treatment. Accordingly, there has been urgently needed to develop new therapeutic agent to prevent and treat the disease nowadays. Many multi-national pharmaceutical companies have been invested on the development in a large scale and in particular, focused in the development for beta- or gamma secretase inhibitor reducing the reproduced amount of beta-amyloid consisting of about 40 amino acids which has been presumed to be an etiological factor of Alzheimer disease. The basic study on the Alzheimer disease has been actively attempted in Korea however the development of Alzheimer treating agent has been merely progressed till now. Since there have been found in animal model test as well as clinical trial that the development of gamma secretase inhibitor is associated with considerable toxicity, it has been proved to be not recommendable whereas the development of beta secretase inhibitor is recommendable as proven by gene deficiency transformed animal model test. It is also regarded as a safe tool to focus on targeting the factors involved in beta amyloid aggregation. There has been reported that ‘phenserine’ developed by Axonyx Co. in USA has been progressed in Clinical trial 2 phase and it shows dual activities of inhibiting cholinesterase as well as beta amyloid aggregation. (Greig et al., J. Med. Chem. 44 pp 4062-4071, 2001; www.medicalnewstoday.com; www.alzforum.org/drg/drc)

The development of vaccine using beta amyloid has been known as another possible method. There has been reported that the serial study on the vaccine progressed by Elan Co. failed because of its unpredictable adverse response such as encephalitis during clinical trial. However, it has been reported that beta amyloid vaccine could alleviate cognitive function in animal model test and improve the activity of brain cell as well as damaged brain neuronal cells, resulting in alleviating Alzheimer syndrome. (Janus et al., Nature 408, pp 979-982, 2000; Morgan et al., Nature 408, pp 982-985, 2000)

There have been reported that the extract of Salviae miltiorrhizae Bunge (Labiatae) can treat various disease, for example, abdominal pain, trauma, insomnia, skin rash etc and contains several diterpene quinine pigments belong to abietanoid compound, for example, tanshinone I, dihydrotanshinone I, tanshinone IIA, tanshinone IIB, methylene tanshinone etc. (Il-Moo Chang et al, Oriental Medicine and Medical Science Encyclopedia, Seoul National University, 2003)

There have been also reported on the pharmacological activity of Salviae miltiorrhizae Bunge (Labiatae), for example, anti-oxidative activity (Yun-Hwa Kim, Korean J. Soc. Food Cookery Sci., 19 p 4, 2003), anti-cancer activity (Ok-Hee Kim, The Journal of Applied Pharmacology, 7, pp 29-34, 1999), lowering effect on blood pressure (Korean Patent No. 10-0327894) etc. However, there has been not reported or disclosed about therapeutic effect on cognitive function disorder of the tanshinone compounds isolated from Salviae miltiorrhizae Bunge (Labiatae) in any of above cited literatures, the disclosures of which are incorporated herein by reference.

To investigate an inhibiting effect of the tanshinone compounds isolated from Salviae miltiorrhizae Bunge (Labiatae) on cognitive function disorder through already well-known screening tests, the inventors of the present invention have intensively screened various plants showing potent inhibiting activity of beta-amyloid aggregation and memory learning recovery study using passive avoidance test etc, and finally completed present invention by confirming that the tanshinone compounds isolated from Salviae miltiorrhizae Bunge (Labiatae) inhibits beta-amyloid aggregation and cell cytotoxicity resulting in stimulating the proliferation of neuronal cells as well as recovers memory learning injury caused by neuronal cell injury

These and other objects of the present invention will become apparent from the detailed disclosure of the present invention provided hereinafter.

### DISCLOSURE

### Technical Problem

Accordingly, it is an object of the present invention to provide a pharmaceutical composition comprising a tanshinone compounds selected from the group consisting of miltirone, 1,2-didehydromiltirone, tanshinone UA, tanshinone I and dihydrotanshinone I isolated from Salviae Miltiorrhizae Bunge as an active ingredient in an effective amount to treat and prevent cognitive function disorder by the mechanism of inhibiting beta-amyloid aggregation and cell cytotoxicity resulting in stimulating the proliferation of neuronal cells as well as recovers memory learning injury caused by neuronal cell injury.

### DISCLOSURE OF THE INVENTION

### Technical Solution

In accordance with the present invention to provide a pharmaceutical composition comprising a tanshinone compounds selected from the group consisting of miltirone, 1,2-didehydromiltirone, tanshinone HA, tanshinone I and dihydrotanshinone I isolated from Salviae Miltiorrhizae Bunge as an active ingredient in an effective amount to treat and prevent cognitive function disorder.

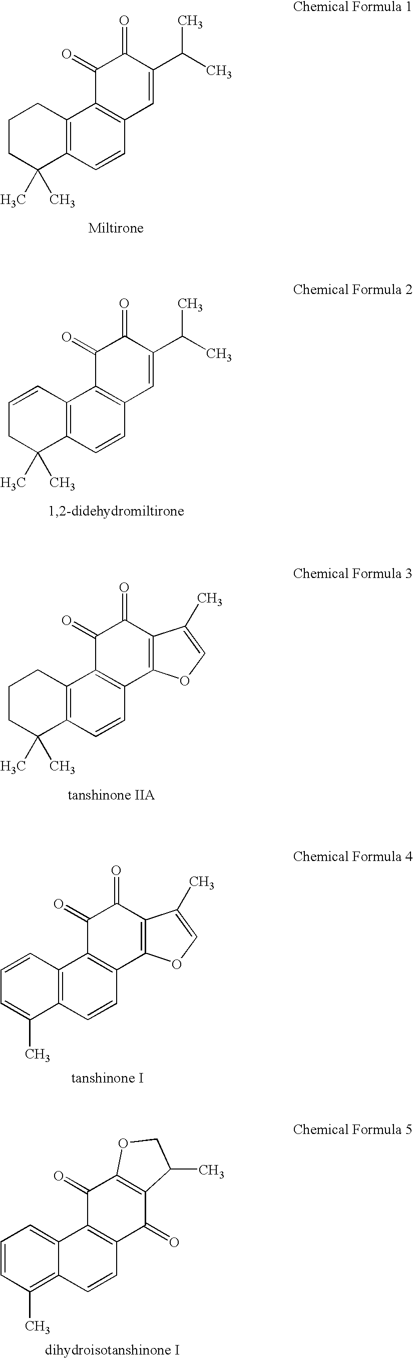

Specifically, the present invention to provide a pharmaceutical composition comprising tanshinone compounds selected from the group consisting of miltirone, 1,2-didehydromiltirone, tanshinone IIA, tanshinone I and dihydrotanshinone I isolated from Salviae miltiorrhizae Bunge as an active ingredient and pharmaceutically acceptable carrier, diluents or adjuvants to treat and prevent cognitive function disorder.

It is an object of the present invention to provide a method of treating or preventing cognitive function disorder in a mammal comprising administering to said mammal an effective amount of a tanshinone compounds selected from the group consisting of miltirone, 1,2-didehydromiltirone, tanshinone IIA, tanshinone I and dihydrotanshinone I isolated from Salviae miltiorrhizae Bunge, together with a pharmaceutically acceptable carrier thereof.

It is an object of the present invention to provide a use of a tanshinone compounds selected from the group consisting of miltirone, 1,2-didehydromiltirone, tanshinone IIA, tanshinone I and dihydrotanshinone I isolated from Salviae miltiorrhizae Bunge for the manufacture of therapeutic agent for the treatment and prevention of cognitive function disorder.

The term “cognitive function disorder’ disclosed herein includes Alzheimer type dementia, cerebrovascular type dementia, pick's disease, Creutzfeldt-jakob's disease, dementia caused by cephalic damage, Parkinson's disease, and so on, preferably, Parkinson's disease.

An inventive tanshinone compounds isolated from Salviae miltiorrhizae Bunge may be prepared in accordance with the following preferred embodiment.

Hereinafter, the present invention is described in detail.

An inventive tanshinone compounds isolated from Salviae miltiorrhizae Bunge can be prepared in detail by following procedures.

The inventive tanshinone compounds isolated from Salviae miltiorrhizae Bunge can be prepared by the procedure comprising the steps consisting of; adding 0.1 to 0.2-fold volume of methanol to dried rhizoma of Salviae miltiorrhizae BGE; extracting with extraction method by the reflux extraction, or ultra-sonication extraction; subjecting the solution with filtering to obtain the supernatant to be concentrated with rotary evaporator, at the temperature ranging from 35 to 45° C.; drying to obtain dried crude extract powder of Salviae miltiorrhizae BGE at 1^{st} step; Suspending said crude extract in distilled water and adding diethylether thereto; mixing and subjecting to fractionation with 3 to 6 times to obtain diethylether soluble fraction of Salviae miltiorrhizae BGE at 2^{nd} step; subjecting the diethyl ether soluble extract to repeating Silica gel column chromatography (Silca gel 60, 70-230 mesh) eluting with solvent mixture (Hexane:ethylacetate) increasing the polarity of the eluting solvent to obtain miltirone, 1,2-didehydromiltirone, tanshinone IIA, tanshinone I and dihydrotanshinone I of the present invention. Also, the above-described procedures may be modified or subjected to further step to fractionate or isolate more potent fractions or compounds by conventional procedure well-known in the art, for example, the procedure disclosed in the literature (Harborne J. B. Phytochemical methods: A guide to modern techniques of plant analysis, 3^{rd} Ed. pp 6-7, 1998).

In accordance with another aspect of the present invention, there is provided a pharmaceutical composition comprising tanshinone compounds selected from the group consisting of miltirone, 1,2-didehydromiltirone, tanshinone IIA, tanshinone I and dihydrotanshinone I isolated from Salviae miltiorrhizae prepared by the above-described preparation method as an active ingredient in an effective amount to treat and prevent cognitive function disorder.

It is an object of the present invention to provide a method of treating or preventing cognitive function disorder in a mammal comprising administering to said mammal an effective amount of tanshinone compounds selected from the group consisting of miltirone, 1,2-didehydromiltirone, tanshinone IIA, tanshinone I and dihydrotanshinone I isolated from Salviae miltiorrhizae prepared by the above-described preparation method, together with a pharmaceutically acceptable carrier thereof.

It is an object of the present invention to provide a use of tanshinone compounds selected from the group consisting of miltirone, 1,2-didehydromiltirone, tanshinone IIA, tanshinone I and dihydrotanshinone I isolated from Salviae miltiorrhizae prepared by above described preparation method for the manufacture of therapeutic agent for the treatment and prevention of cognitive function disorder.

The pharmaceutical composition of the present invention can contain about 0.01˜50% by weight of the above extract based on the total weight of the composition.

Through various screening test to determine inhibiting effect of tanshinone compounds selected from the group consisting of miltirone, 1,2-didehydromiltirone, tanshinone IIA, tanshinone I and dihydrotanshinone I isolated from Salviae miltiorrhizae on cognitive function disorder, it has confirmed that the tanshinone compounds selected from the group consisting of miltirone, 1,2-didehydromiltirone, tanshinone IIA, tanshinone I and dihydrotanshinone I isolated from Salviae miltiorrhizae inhibit beta-amyloid aggregation as well as the toxicity and cell apoptosis caused by beta amyloid resulting in stimulating the proliferation of neuronal cells and moreover recover memory learning injury caused by neuronal cell injury.

The inventive composition for treating and preventing cognitive function disorder may comprises the above compound as 0.01˜50% by weight based on the total weight of the composition.

The inventive composition may additionally comprise conventional carrier, adjuvants or diluents in accordance with a using method well known in the art. It is preferable that said carrier is used as appropriate substance according to the usage and application method, but it is not limited. Appropriate diluents are listed in the written text of Remington's Pharmaceutical Science (Mack Publishing co, Easton Pa.).

Hereinafter, the following formulation methods and excipients are merely exemplary and in no way limit the invention.

The composition according to the present invention can be provided as a pharmaceutical composition containing pharmaceutically acceptable carriers, adjuvants or diluents, e.g., lactose, dextrose, sucrose, sorbitol, mannitol, xylitol, erythritol, maltitol, starches, acacia rubber, alginate, gelatin, calcium phosphate, calcium silicate, cellulose, methyl cellulose, polyvinyl pyrrolidone, water, methylhydroxy benzoate, propylhydroxy benzoate, talc, magnesium stearate and mineral oil. The formulations may additionally include fillers, anti-agglutinating agents, lubricating agents, wetting agents, flavoring agents, emulsifiers, preservatives and the like. The compositions of the invention may be formulated so as to provide quick, sustained or delayed release of the active ingredient after their administration to a patient by employing any of the procedures well known in the art.

For example, the compositions of the present invention can be dissolved in oils, propylene glycol or other solvents that are commonly used to produce an injection. Suitable examples of the carriers include physiological saline, polyethylene glycol, ethanol, vegetable oils, isopropyl myristate, etc., but are not limited to them. For topical administration, the extract of the present invention can be formulated in the form of ointments and creams.

Pharmaceutical formulations containing present composition may be prepared in any form, such as oral dosage form (powder, tablet, capsule, soft capsule, aqueous medicine, syrup, elixirs pill, powder, sachet, granule), or topical preparation (cream, ointment, lotion, gel, balm, patch, paste, spray solution, aerosol and the like), or injectable preparation (solution, suspension, emulsion).

The composition of the present invention in pharmaceutical dosage forms may be used in the form of their pharmaceutically acceptable salts, and also may be used alone or in appropriate association, as well as in combination with other pharmaceutically active compounds.

The desirable dose of the inventive compound or composition varies depending on the condition and the weight of the subject, severity, drug form, route and period of administration, and may be chosen by those skilled in the art. However, in order to obtain desirable effects, it is generally recommended to administer at the amount ranging 10 g/kg, preferably, 1 to 3 g/kg by weight/day of the inventive extract or compounds of the present invention. The dose may be administered in single or divided into several times per day. In terms of composition, the amount of inventive compound should be present between 0.01 to 50% by weight, preferably 0.5 to 40% by weight based on the total weight of the composition.

The pharmaceutical composition of present invention can be administered to a subject animal such as mammals (rat, mouse, domestic animals or human) via various routes. All modes of administration are contemplated, for example, administration can be made orally, rectally or by intravenous, intramuscular, subcutaneous, intracutaneous, intrathecal, epidural or intracerebroventricular injection.

It is another object of the present invention to provide a health food or food additives comprising tanshinone compounds selected from the group consisting of miltirone, 1,2-didehydromiltirone, tanshinone IIA, tanshinone I and dihydrotanshinone I isolated from Salviae miltiorrhizae BGE, together with a sitologically acceptable additive for the prevention and improvement of cognitive function disorder.

The health food of the present invention comprises the above compounds as 0.01 to 80%, preferably 1 to 50% by weight based on the total weight of the composition.

Above health food can be contained in health food, health beverage etc, and may be used as powder, granule, tablet, chewing tablet, capsule, beverage etc.

Also, the present invention provide a composition of the health food beverage for the prevention and improvement of cognitive function disorder adding the above described compound 0.01 to 80% by weight, amino acids 0.001 to 5% by weight, vitamins 0.001 to 2% by weight, sugars 0.001 to 20% by weight, organic acids 0.001 to 10% by weight, sweetener and flavors of proper amount.

To develop for health food, examples of addable food comprising the above compounds of the present invention are various food, beverage, gum, vitamin complex, health improving food and the like, and can be used as power, granule, tablet, chewing tablet, capsule or beverage etc.

Also, the compound of the present invention will be able to prevent and improve cognitive function disorder by way of adding to child and infant food, such as modified milk powder, modified milk powder for growth period, modified food for growth period.

Above described composition therein can be added to food, additive or beverage, wherein, the amount of the above-described compound in food or beverage may generally range from about 0.1 to 80 w/w %, preferably 1 to 50 w/w % of total weight of food for the health food composition and 1 to 30 g, preferably 3 to 10 g on the ratio of 100 ml of the health beverage composition.

Providing that the health beverage composition of present invention contains the above-described compound as an essential component in the indicated ratio, there is no particular limitation on the other liquid component, wherein the other component can be various deodorant or natural carbohydrate etc such as conventional beverage. Examples of aforementioned natural carbohydrate are monosaccharide such as glucose, fructose etc; disaccharide such as maltose, sucrose etc; conventional sugar such as dextrin, cyclodextrin; and sugar alcohol such as xylitol, and erythritol etc. As the other deodorant than aforementioned ones, natural deodorant such as taumatin, stevia extract such as levaudioside A, glycyrrhizin et al., and synthetic deodorant such as saccharin, aspartam et al., may be useful favorably. The amount of above described natural carbohydrate is generally ranges from about 1 to 20 g, preferably 5 to 12 g in the ratio of 100 ml of present beverage composition.

The other components than aforementioned composition are various nutrients, a vitamin, a mineral or an electrolyte, synthetic flavoring agent, a coloring agent and improving agent in case of cheese chocolate et al., pectic acid and the salt thereof, alginic acid and the salt thereof, organic acid, protective colloidal adhesive, pH controlling agent, stabilizer, a preservative, glycerin, alcohol, carbonizing agent used in carbonate beverage et al. The other component than aforementioned ones may be fruit juice for preparing natural fruit juice, fruit juice beverage and vegetable beverage, wherein the component can be used independently or in combination. The ratio of the components is not so important but is generally range from about 0 to 20 w/w % per 100 w/w % present composition. Examples of addable food comprising aforementioned extract therein are various food, beverage, gum, vitamin complex, health improving food and the like.

The inventive composition may additionally comprise one or more than one of organic acid, such as citric acid, fumaric acid, adipic acid, lactic acid, malic acid; phosphate, such as phosphate, sodium phosphate, potassium phosphate, acid pyrophosphate, polyphosphate; natural anti-oxidants, such as polyphenol, catechin, α-tocopherol, rosemary extract, vitamin C, green tea extract, licorice root extract, chitosan, tannic acid, phytic acid etc.

The above described compound isolated from Salviae miltiorrhizae BGE may be 20 to 90% high concentrated liquid, power, or granule type.

Similarly, the above compound isolated from Salviae miltiorrhizae BGE can comprise additionally one or more than one of lactose, casein, dextrose, glucose, sucrose and sorbitol.

Inventive compound of the present invention have no toxicity and adverse effect therefore; they can be used with safe.

It will be apparent to those skilled in the art that various modifications and variations can be made in the compositions, use and preparations of the present invention without departing from the spirit or scope of the invention.

### ADVANTAGEOUS EFFECTS

As described in the present invention, the tanshinone compounds isolated from the extract of Salviae Miltiorrhizae Bunge (Labiatae) inhibits beta-amyloid aggregation as well as the toxicity and cell apoptosis caused by beta amyloid resulting in stimulating the proliferation of neuronal cells, Therefore, it can be used as the therapeutics or health food for treating and preventing cognitive function disorder without adverse action.

### DESCRIPTION OF THE DRAWINGS

The above and other objects, features and other advantages of the present invention will more clearly understood from the following detailed description taken in conjunction with the accompanying drawings, in which.

FIG. 1 shows the isolation scheme for tanshinone compounds from the extract of Salviae miltiorrhizae BGE.

FIG. 2 shows the inhibitory effect of miltirone (compound S-2-3) on the aggregation and cytotoxicity of beta amyloid.

FIG. 3 shows the inhibitory effect of didehydromiltirone (compound S-2-6) on the aggregation and cytotoxicity of beta amyloid.

FIG. 4 represents the inhibitory effect of tanshinone IIA (compound S-4-4-1) on the aggregation and cytotoxicity of beta amyloid.

FIG. 5 represents the inhibitory effect of tanshinone I (compound S-8-4) on the aggregation and cytotoxicity of beta amyloid.

FIG. 6 represents the inhibitory effect of dihydrotanshinone I (compound S-8-11) on the aggregation and cytotoxicity of beta amyloid;

FIG. 7 presents the result of memory learning study (Y maze test) using tanshinone IIA (compound S-4-4-1).

FIG. 8 depicts the result of memory learning study (PA test) using tanshinone IIA (compound S-4-4-1).

FIG. 9 depicts comparison of mouse brain staining between control group and test group treated with tanshinone IIA (compound S-4-4-1).

### BEST MODE

The present invention is more specifically explained by the following examples. However, it should be understood that the present invention is not limited to these examples in any manner.

The following Reference Example, Examples and Experimental Examples are intended to further illustrate the present invention without limiting its scope.

### Example 1

### Preparation of the Tanshinone Compounds Isolated from the Extract of Salviae miltiorrhizae

### 1-1. Preparation of Methanol Soluble Extract

15 kg of dried of Salviae miltiorrhizae BGE purchased from Kyung-dong Market located in Seoul was cut into small pieces, mixed with 2.5 L of methanol and subjected to reflux-extraction for 3 hours at three times. The residue is filtered to obtain the supernatant and the filtrate was dried with vacuum evaporator at 40° C. to obtain 3.4 kg of methanol soluble extract.

### 1-2. Preparation of Diethyl Ether Soluble Extract

3.4 kg of methanol soluble extract was suspended in distilled water and diethylether solvent was added thereto. The suspension was subject to fractionation at 3 to 4 times to obtain water soluble extract and diethyl ether soluble extract. The diethylether soluble extract was concentrated and dried to obtain 300 g of diethyl ether soluble extract of Salviae miltiorrhizae BGE.

### 1-3. Preparation of the Tanshinone Compounds

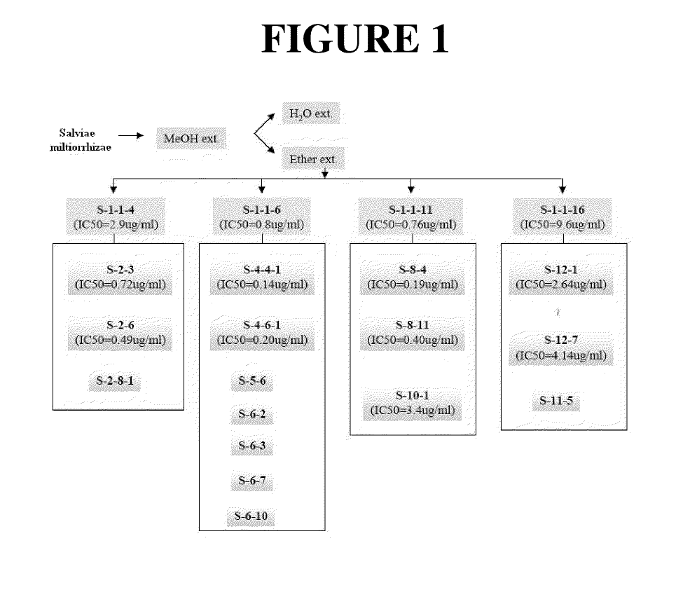

As shown in FIG. 1, 300 g of diethyl ether soluble extract of Salviae miltiorrhizae BGE was loaded to Silica gel column chromatography (Silica gel 60, 70-230 mesh) eluting with solvent mixture (hexane:ethylacetate=20:1) 3 to obtain 15 purified fractions. To purify further, the 4^{th} fraction among said fractions was loaded to Silica gel column chromatography (Silica gel 60, 70-230 mesh) eluting with solvent mixture (hexane:ethylacetate=25:1) to obtain miltirone and 1,2-didehydromiltirone. the 6^{th} fraction among said fractions was loaded to Silica gel column chromatography (Silica gel 60, 70-230 mesh) eluting with solvent mixture (hexane:ethylacetate=15:1) and subjected to recrystallization with dichloromethane to obtain tanshinone IIA. the 11^{th} fraction among said fractions was loaded to Silica gel column chromatography (Silica gel 60, 70-230 mesh) eluting with solvent mixture (hexane:acetone=20:1) and subjected to recrystallization with dichloromethane to obtain tanshinone I and dihydroisotanshinone I. The determined physicochemical property of each compound was shown as follows:

### Chemical Formula 1

### Miltirone

1). Molecular Formula: C₁₉H₂₂O₂

2). Molecular Weight: 282

3). ¹H-NMR (500 MHz, CDCl₃, ppm): 7.59 (1H, d, J=7.9 Hz), 7.11 (1H, d, J=7.9 Hz), 7.07 (1H, s), 3.18 (2H, t, J=6.4 Hz), 3.02 (1H, sept, J=6.8 Hz), 1.79 (2H, m), 1.65 (2H, m), 1.30 (6H, s), 1.16 (6H, d, J=6.9 Hz)

4). ¹³C-NMR (125 MHz, CDCl₃, ppm): 182.5, 181.7, 149.8, 145.2, 144.4, 140.0, 134.6, 133.9, 128.4, 128.0, 38.0, 34.6, 31.9, 30.0, 27.0, 21.7, 19.2

### Chemical Formula 2

1,2-didehydromiltirone

1). Molecular Formula: C₁₉H₂₀O₂

2). Molecular Weight: 280

3). ¹H-NMR (500 MHz, CDCl₃, ppm): 7.85 (1H, d, J=10.1 Hz), 7.09-7.49 (2H, ABq, J=7.7 Hz), 7.08 (1H, s), 6.31 (1H, td, J=5, 10 Hz), 3.01 (1H, sept, J=7.1 Hz), 2.26 (2H, d, J=4.5 Hz), 1.27 (6H, s), 1.15 (6H, d, J=7 Hz)

4). ¹³C-NMR (125 MHz, CDCl₃, ppm): 183.3, 181.6, 148.1, 145.0, 140.1, 137.3, 134.6, 134.4, 130.7, 129.4, 124.8, 38.5, 34.1, 29.8, 28.5, 27.0. 21.6

### Chemical Formula 3

tanshinone IIA

1). Molecular Formula: C₁₉H₁₈O₃

2). Molecular Weight: 294

3). ¹H-NMR (500 MHz, CDCl₃, ppm): 7.64 (1H, d, J=8 Hz), 7.56 (1H, d, J=8 Hz), 7.23 (1H, d, J=1 Hz), 3.20 (2H, t, J=6.4 Hz), 2.28 (3H, d, J=1 Hz), 1.80 (2H, m), 1.66 (2H, m), 1.32 (6H, s)

4). ¹³C-NMR (125 MHz, CDCl₃, ppm): 183.6, 175.7, 171.7, 150.1, 144.1, 141.1, 133.3, 127.4, 126.5, 121.4, 120.8, 119.9, 37.8, 34.7, 31.2, 29.9, 19.1, 17.9

### Chemical Formula 4

tanshinone I

1). Molecular Formula: C₁₈H₁₂O₃

2). Molecular Weight: 276

3). ¹H-NMR (500 MHz, CDCl₃, ppm): 9.23 (1H, d, J=8 Hz), 8.27 (1H, d, J=8 Hz), 7.77 (1H, d, J=8 Hz), 7.27-7.55 (3H, m), 2.68 (3H, s), 2.29 (3H, s)

4). ¹³C-NMR (125 MHz, CDCl₃, ppm): 183.6, 175.8, 161.4, 142.3, 135.4, 133.8, 133.1, 132.9, 130.8, 129.8, 128.6, 125.0, 123.3, 121.9, 120.7, 118.9, 20.1, 9.0

### Chemical Formula 5

dihydroisotanshinone I

1). Molecular Formula: C₁₈H₁₄O₃

2). Molecular Weight: 278

3). ¹H-NMR (500 MHz, CDCl₃, ppm): 9.28 (1H, d, J=8 Hz), 7.72-8.28 (2H, q, J=8 Hz), 7.39-7.58 (2H, m), 4.97 (1H, t, J=9 Hz), 4.43 (1H, dd, J=9, 6 Hz), 3.63-3.68 (1H, m), 2.69 (3H, s), 1.42 (3H, d, J=7 Hz),

4). ¹³C-NMR (125 MHz, CDCl₃, ppm): 184.5, 175.9, 170.7, 135.2, 134.9, 132.3, 132.1, 130.6, 129.0, 128.4, 126.3, 125.1, 120.5, 118.6, 81.8, 34.9, 20.0, 19.0

### Experimental Example 1

### In Vitro Activity Test

### 1-1. Preparation of Experiment

### 1-1-1. Inhibition Test of Beta Amyloid Aggregation

Synthetic beta amyloid 1-42 (BACHEM) was dissolved in DMSO in order to 250 μM solution and diluted with PBS into 1/10 on fluorescent black plate to induce aggregation. By comparing with inhibition activity of the tanshinone compounds prepared in Example 1 on beta amyloid aggregation, the test sample showing more than 50% inhibition activity at 10 μg/ml was chosen to use and added to react for 1 hour at room temperature. ThT (Thioflavin T) was diluted with 50 mM glycine buffer solution and the diluted solution was added to each well by 150 μl/well. The absorbance was determined by microplate reader (SAFIRE, TECAN) at 450 nm excitation wavelength/480 nm emission wavelength and the inhibition activity of the test sample on beta amyloid aggregation was transformed into IC₅₀.

### 1-1-2. Inhibition Test of Beta Amyloid Toxicity

HT 22 mouse neuronal cell line was incubated in DMEM (Dulbecco's Modified Eagle's Medium, Gibco-BRL) medium supplemented with 10% FBS (Fetal Bovine Serum, Hyclone) and 1% penicillin/streptomycin (Sigma Co.) Prior to test, HT22 cell was incubated on 96 well plates with a density of 5×10³ cell/well and further incubated in serum free DMEM medium for 1 hour before the treatment of test sample. Various concentration of diethyl ether extract prepared in Example 1 used as a test sample was added thereto and incubated for 1 hour. Aggregated beta amyloid (US peptide) was treated thereto to the concentration of 25 μM and incubated for 18 hours to induce cell necrosis. 5 mg/ml of MTT (3-(4,5-dimethyl-2-thiazolyl)-2,5-diphenyl-2H-tetrazolium bromide) solution was added each well with 15 μl/well and the well was incubated for 4 hours. Dissolving buffer solution (10% SDS, 50% dimethyl formamide, pH 4.7) was added to each well with 100 μl/well and reacted for overnight. 18 hours after the reaction, the absorbance of solution was determined by microplate reader (SAFIRE, TECAN) at 570 nm/630 nm wavelength (Gillardon, F. et al., Brain Research, 706(1) pp 169-172, 1996).

### 1-1-3. Determination of Cytotoxicity

To determine the toxicity of test sample, HT22 cell was incubated in accordance with similar method disclosed in 1-1-2 and various concentration of the test sample prepared in Example 1 was added to the cell to incubate for 18 hours. MTT solution and Dissolving buffer solution was added to cell serially and the absorbance was determined by microplate reader (SAFIRE, TECAN) at 570 nm.

### 1-2. Activity Result of Miltirone (Compound S-2-3)

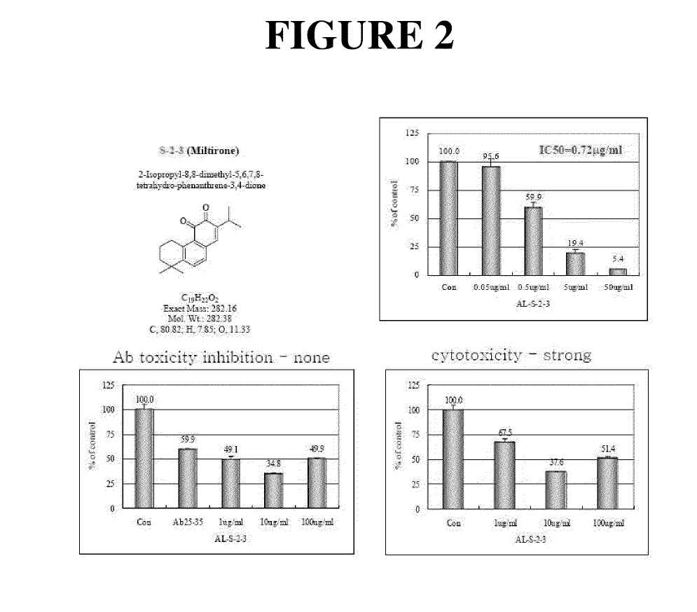

As can be shown in FIG. 2, Miltirone (compound S-2-3) showed potent inhibitory effect on the aggregation of beta amyloid (0.72 mg/ml) while it did not inhibit the toxicity of beta amyloid.

### 1-3. Activity Result of 1,2-Didehydromiltirone (Compound S-2-6)

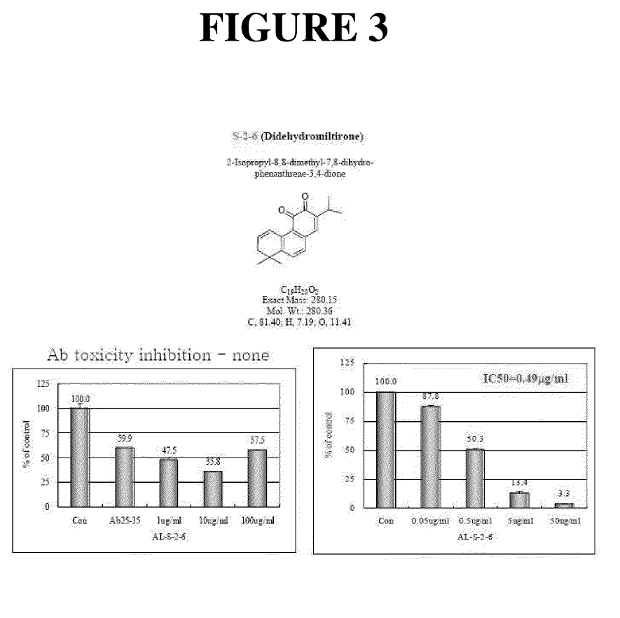

As can be shown in FIG. 3, 1,2-Didehydromiltirone (compound S-2-6) showed potent inhibitory effect on the aggregation of beta amyloid (0.49 mg/ml) while it did not inhibit the toxicity of beta amyloid.

### 1-4. Activity Result of Tanshinone IIA (Compound S-4-4-1)

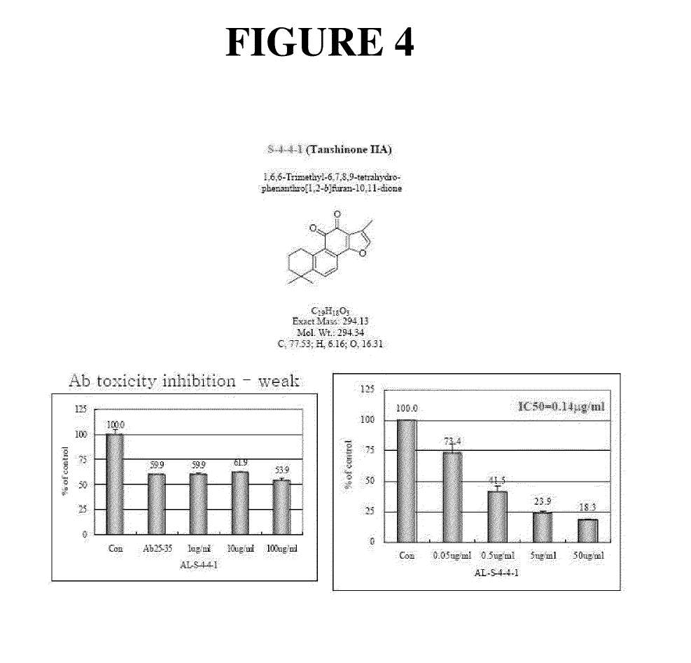

As can be shown in FIG. 4, tanshinone IIA (compound S-4-4-1) showed most potent inhibitory effect on the aggregation of beta amyloid among the test samples (0.14 mg/ml) and showed mere inhibitory effect on the toxicity of beta amyloid.

### 1-5. Activity Result of Tanshinone I (Compound S-8-4)

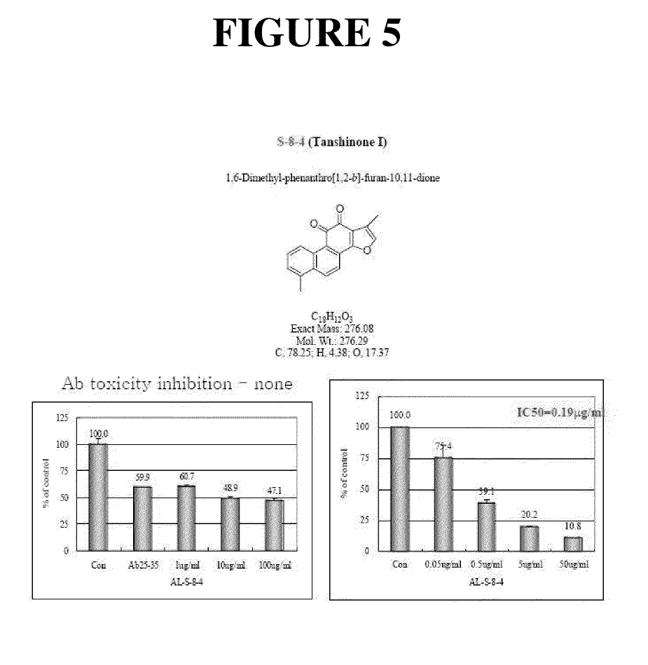

As can be shown in FIG. 5, tanshinone I (compound S-8-4) showed potent inhibitory effect on the aggregation of beta amyloid (0.19 mg/ml) and did not show any inhibitory effect on the toxicity of beta amyloid.

### 1-6. Activity Result of Dihydrotanshinone I (Compound S-8-11)

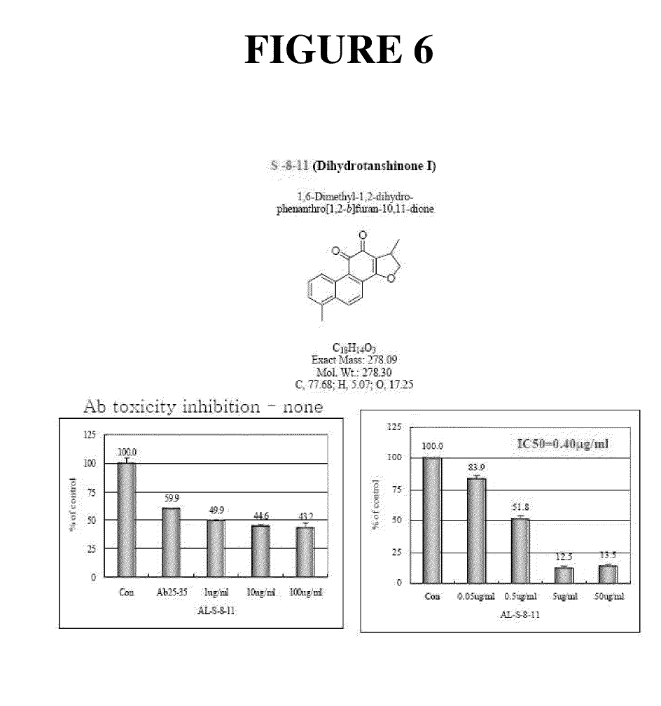

As can be shown in FIG. 6, dihydrotanshinone I (compound S-8-11) showed potent inhibitory effect on the aggregation of beta amyloid (0.40 mg/ml) and did not show any inhibitory effect on the toxicity of beta amyloid.

### Experimental Example 2

### In Vivo Activity Test

### 2-1. Experimental Design

For passive avoidance test, male ICR mouse weighing 25 g purchased from Samtaco Co. was bred with five mice per cage and the cage was kept with following condition maintaining the temperature of 22±2° C. and the relative humidity of 50±5° C. under the regularly controlled light/dark condition with an interval of 12 hours.

Synthetic beta amyloid 1-42 (BACHEM) was dissolved in DMSO in order to be 250 μM solution and diluted with PBS to 10 nM and aggregated at 37° C. for four days (Passive Avoidance test) or six days (Y maze test).

Aggregated beta amyloid 1-42 was administrated into the mice according to the procedure disclosed in the literature (Lausen & Belknap, J. Pharmacol. Methods, 16 pp 355-357, 1986).

50 μl of aggregated beta amyloid 1-42 was administrated into the 2.4 mm depth of bregma region with 50 μl of Hamilton micro-syringe equipped with 26-gauge needle. The behavior tests were divided into Y maze test and PA (passive avoidance) test after the beta amyloid administration. Y maze test was performed 2 days after the administration and PA test was 3 days after the administration. Each test was done with more than 10 mice.

At the end of the experiment, the brain of animals was delivered and kept in 10% formalin solution to staining.

### 2-2. Drug Treatment

After the administration of beta amyloid, the tanshinone compounds prepared in Example 1 were administrated into the mice at the interval of once a day in case of Y maze test and the test samples were continuously administrated for three days in case of passive avoidance test dividing into two test groups, i.e., one is 50 mg/kg treatment group and another group is 100 mg/kg treatment group. Each test was performed at the next day of the administration.

### 2-3. Behavior Procedure

### 2-3-1. AD Acute Model Experiment—Y Maze Test

Y-maze test was performed two days after the administration of beta amyloid. Black acrylic Y maze box consists of three arms (length: 40 cm, Height: 10 cm, Width 5 cm) having identical angle between each other. The mice were positioned at the center of maze and let to move freely for eight minutes with the maze. The entering order of mice in the pathway was observed and the entering latency time was determined when four limbs was entered within the pathway. To determine the spatial memory, the determined spontaneous alteration behavior was calculated by following empirical formula 1 and the actual alteration was assigned to one time at the time that mice was entered three pathways continuously.

Spontaneous alteration (%)=[actual alteration/total arm entries-2]×100 Math Figure 1

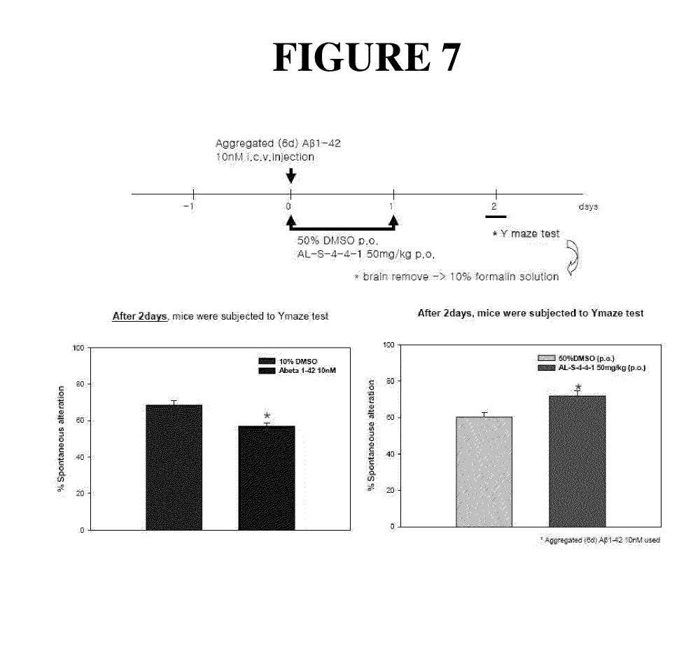

Since tanshinone IIA (compound S-4-4-1) showed most potent inhibitory effect on the aggregation of beta amyloid among the test samples and no toxicity at oral administration test (2000 mg/kg), 50 mg/kg of the compound was orally administrated into the mice directly treated with beta amyloid through intracerebro-ventricular pathway and the recovering effect of the compound on brain damage was found through Y maze test. The treatment of 100 mg/kg of tanshinone IIA also showed similar effect to that of 100 mg/kg of tanshinone IIA. As can be seen in FIG. 7, the mice showed memory learning disorder caused by beta amyloid administration recovered by the treatment of tanshinone IIA (compound S-4-4-1).

### 2-3-2. AD Acute Model Experiment—Passive Avoidance Test

Passive Avoidance test was performed three days after the administration of beta amyloid. Black avoidance shuttle box was divided into two chambers of equal size (18 cm L×9.5 cm W×17 cm H) partitioned by compartment door (4 cm L×3.5 cm W) allowing electricity to run on the floor of the dark compartment. A light chamber is equipped with a 20-W lamp on the hinged plexiglass lid and the mice were allowed to enter dark chamber through compartment door.

The experiments consisted of training and test sessions.

In the training session, male mice weighing 25 g were initially placed in the light chamber and allowed for habituation. The door was then opened and as soon as mice preferring darkness went out from light chamber and entered the dark chamber the door was closed immediately, and an electric shock (0.25 mA, 3s, once) was delivered to the mouse through the grid floor for 3 sec.

At 24 hours after the training session, the identical experiment was performed again with mouse to measure the latency time staying at the light chamber. The data was regarded as the index which meant the memory on previous training by 0.25 mA of electronic shock for 3 second. Latency to enter the dark compartment from the light compartment was measured as a step through latency. If it did not enter the dark chamber within the cut-off time (300 sec), it was assigned a value of 300 sec as its latency. Passive avoidance test was performed using by the mice orally administrated with test sample prepared in Example.

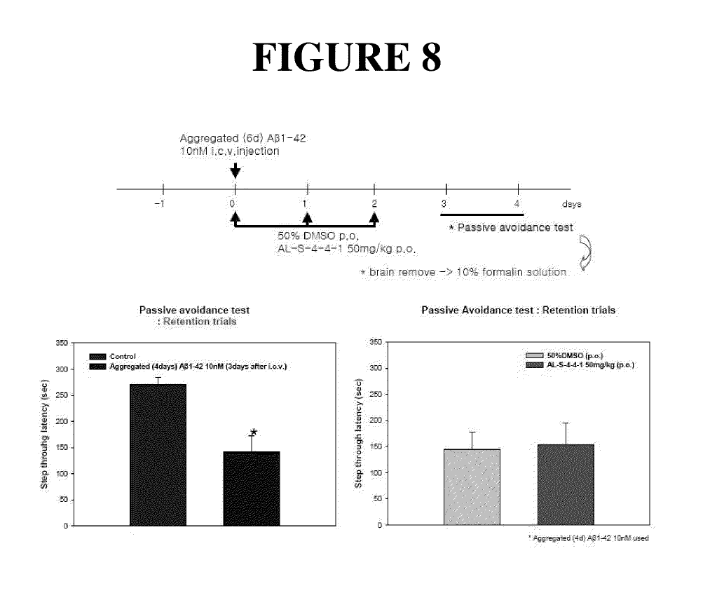

As shown in the FIG. 8, the change of latency time means the decline or recovery of memory and the lengthened latency time means the increased memory. In the sham operated control group, there was no change in latency time and in the vehicle control group administered with solvent, the latency time treated with tanshinone IIA (compound S-4-4-1) significantly increased compared with the sham control group (p<0.05).

### 2-3-3. Immunochemistry Brain Staining of AD Acute Model Experiment

At the end of behavior test, the mouse brain was delivered, kept in 10% formalin solution for 24 hours and transferred to 30% sucrose solution. After fixing the brain, the brain was performed to coronal section with a width of 40 μm using by cryostat. The sliced brain was performed to Cresyl violet staining to confirm the injury of brain neuronal cell, to ChAT staining to confirm the injury of cholinergic neuron and to GFAP staining to confirm the activation of astrocytes.

### (a) Cresyl Violet Staining

After the tissue was placed on gelatin-coated slide to stain with Cresyl violet, the tissue was performed to dehydration using ethanol. The tissue was incubated for about 3 minutes and dipped into 0.5% Cresyl Violet solution for 30 mins. After the solution was performed to re-hydration with ethanol, the slice was dipped into xylene for 3 minutes. The dried tissue was fixed with Canada balsam mounting medium.

### (b) Immunohistochemistry

In the washing process between all the antibody incubation, PBST was used to washing tissues. To reduce the activity of endogenous peroxidase activity, the tissue was pretreated with 0.5% H2O2 and then treated with 5% FBS at room temperature for 1 hour to remove non-specific binding. The tissue was incubated at 4° C. for overnight using by mouse anti-GFAP (1:200) monoclonal antibody and goat-anti-ChAT (1:200) polyclonal antibody. A horse radish peroxidase-conjugated anti-mouse IgG and anti-goat IgG secondary antibody (1:600) was incubated at room temperature for 1 hour and detected by DAB kit after the incubation.

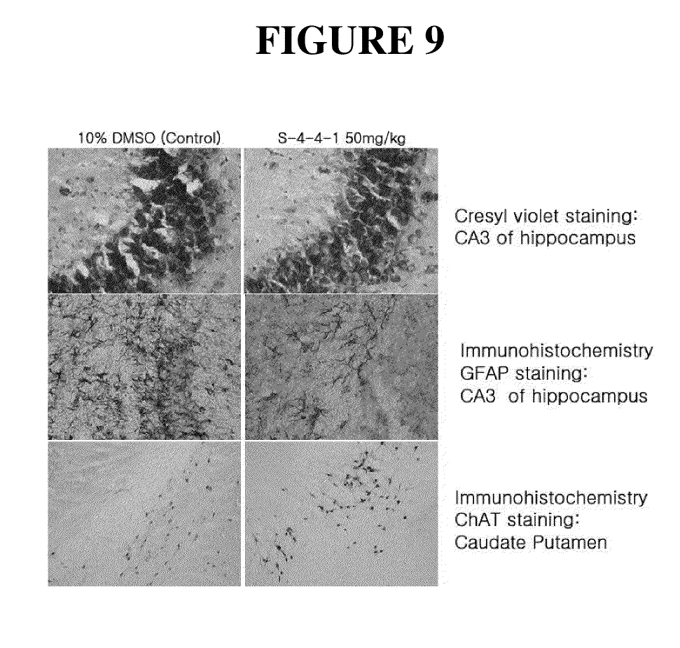

As can be shown in FIG. 9, it has been confirmed that the injury of memory learning caused by beta amyloid is closely correlated with the injury of the specific region of hippocampus and tanshinone IIA (compound S-4-4-1) significantly recovered the injury of memory learning. At the result of ChAT and GFAP staining, tanshinone IIA (compound S-4-4-1) recovered the injury of cholinergic neuron caused by beta amyloid and reduced the increased activity of astrocyte correlated with inflammatory response caused by beta amyloid.

Hereinafter, the formulating methods and kinds of excipients will be described, but the present invention is not limited to them. The representative preparation examples were described as follows.

Preparation of powder

| | |
|---|---|
| Miltirone | 50 mg |
| Lactose | 100 mg |
| Talc | 10 mg |

Powder preparation was prepared by mixing above components and filling sealed package.

Preparation of tablet

| | | |
|---|---|---|
| 1,2-didehydromiltirone | 50 | mg |
| Corn Starch | 100 | mg |
| Lactose | 100 | mg |
| Magnesium Stearate | 2 | mg |

Tablet preparation was prepared by mixing above components and entabletting.

Preparation of capsule

| | | |
|---|---|---|
| Tanshinone IIA | 50 | mg |
| Corn starch | 100 | mg |
| Lactose | 100 | mg |
| Magnesium Stearate | 2 | mg |

Tablet preparation was prepared by mixing above components and filling gelatin capsule by conventional gelatin preparation method.

Preparation of injection

| | |
|---|---|
| Tanshinone I | 50 mg |
| Distilled water for injection | optimum amount |
| PH controller | optimum amount |

Injection preparation was prepared by dissolving active component, controlling pH to about 7.5 and then filling all the components in 2 ml ample and sterilizing by conventional injection preparation method.

Preparation of liquid

| | | |
|---|---|---|
| Dihydroisotanshinone | 0.1~80 | g |
| Sugar | 5~10 | g |
| Citric acid | 0.05~0.3% | |
| Caramel | 0.005~0.02% | |
| Vitamin C | 0.1~1% | |
| Distilled water | 79~94% | |
| CO₂ gas | 0.5~0.82% | |

Liquid preparation was prepared by dissolving active component, filling all the components and sterilizing by conventional liquid preparation method.

Preparation of health food

| Tanshinone IIA | 1000 | mg |
|---|---|---|
| Vitamin mixture | optimum amount | |
| Vitamin A acetate | 70 | μg |
| Vitamin E | 1.0 | mg |
| Vitamin B₁ | 0.13 | mg |
| Vitamin B₂ | 0.15 | mg |
| Vitamin B6 | 0.5 | mg |
| Vitamin B12 | 0.2 | μg |
| Vitamin C | 10 | mg |
| Biotin | 10 | μg |
| Amide nicotinic acid | 1.7 | mg |
| Folic acid | 50 | μg |
| Calcium pantothenic acid | 0.5 | mg |
| Mineral mixture | optimum amount | |
| Ferrous sulfate | 1.75 | mg |
| Zinc oxide | 0.82 | mg |
| Magnesium carbonate | 25.3 | mg |
| Monopotassium phosphate | 15 | mg |
| Dicalcium phosphate | 55 | mg |
| Potassium citrate | 90 | mg |
| Calcium carbonate | 100 | mg |
| Magnesium chloride | 24.8 | mg |

The above-mentioned vitamin and mineral mixture may be varied in may ways. Such variations are not to be regarded as a departure from the spirit and scope of the present invention.

Preparation of health beverage

| | | |
|---|---|---|
| Miltirone | 1000 | mg |
| Citric acid | 1000 | mg |
| Oligosaccharide | 100 | g |
| Apricot concentration | 2 | g |
| Taurine | 1 | g |
| Distilled water | 900 | ml |

Health beverage preparation was prepared by dissolving active component, mixing, stirred at 85° C. for 1 hour, filtered and then filling all the components in 1000 ml ample and sterilizing by conventional health beverage preparation method.

The invention being thus described, it will be obvious that the same may be varied in many ways. Such variations are not to be regarded as a departure from the spirit and scope of the present invention, and all such modifications as would be obvious to one skilled in the art are intended to be included within the scope of the following claims.

### INDUSTRIAL APPLICABILITY

As described in the present invention, the tanshinone compounds isolated from the extract of Salviae miltiorrhizae Bunge (Labiatae) inhibits beta-amyloid aggregation as well as the toxicity and cell apoptosis caused by beta amyloid resulting in stimulating the proliferation of neuronal cells, Therefore, it can be used as the therapeutics or health food for treating and preventing cognitive function disorder without adverse action.

1. A pharmaceutical composition comprising tanshinone compounds selected from the group consisting of miltirone, 1,2-didehydromiltirone, tanshinone IIA, tanshinone I and dihydrotanshinone I isolated from Salviae miltiorrhizae Bunge as an active ingredient and pharmaceutically acceptable carrier, diluents or adjuvants to treat and prevent cognitive function disorder.

2. The pharmaceutical composition according to claim 1 wherein said cognitive function disorder is selected from Alzheimer type dementia, cerebrovascular type dementia, pick's disease, Creutzfeldt-jakob's disease, dementia caused by cephalic damage or Parkinson's disease.

3. A method of treating or preventing cognitive function disorder in a mammal comprising administering to said mammal an effective amount of tanshinone compounds selected from the group consisting of miltirone, 1,2-didehydromiltirone, tanshinone IIA, tanshinone I and dihydrotanshinone I isolated from Salviae miltiorrhizae Bunge, together with a pharmaceutically acceptable carrier thereof.

4. (canceled)

5. A health food comprising tanshinone compounds selected from the group consisting of miltirone, 1,2-didehydromiltirone, tanshinone IIA, tanshinone I and dihydrotanshinone I isolated from Salviae miltiorrhizae Bunge, together with a sitologically acceptable additive for the prevention and improvement of cognitive function disorder.

6. The health food according to claim 5 wherein said health food is provided as powder, granule, tablet, chewing tablet, capsule or beverage type.
